(19) United States
(12) Patent Application Publication
(10) Pub. No.: US 2011/0105897 A1
(43) Pub. Date: May 5, 2011

(54) HYBRID MEDICAL DEVICE LOCALIZATION SYSTEM

(76) Inventors: Giora Kornblau, Binyamina (IL); David Maier Neustadter, Nof Ayalon (IL); Saul Stokar, Raanana (IL)

(21) Appl. No.: 12/937,882

(22) PCT Filed: Apr. 6, 2009
(86) PCT No.: PCT/IL09/00383

§ 371 Date: Jan. 18, 2011

Related U.S. Application Data
(60) Provisional application No. 61/045,270, filed on Apr. 15, 2008.

(51) Int. Cl. A61B 6/00

(52) U.S. Cl. 600/436

(57) ABSTRACT

A hybrid radioactive and impedance tracking system for tracking a target in a human or animal body, comprising: a) a radioactive tracking subsystem for tracking the target; b) an impedance tracking subsystem for tracking the target; and c) a processor that uses data from the radioactive tracking subsystem in combination with data from the impedance tracking system to estimate a position of the target.

### RELATED APPLICATION

The present application claims benefit under 35 USC 119(e) from U.S. provisional patent application 61/045,270, filed on Apr. 15, 2008.

The contents of the above document are incorporated by reference as if fully set forth herein.

### FIELD AND BACKGROUND OF THE INVENTION

The present invention, in some embodiments thereof, relates to a hybrid radioactive and impedance tracker and, more particularly, but not exclusively, to a hybrid tracker used to track the position of a medical device in the body.

Medical tracking devices are commonly used in a wide range of medical applications such as treating cardiovascular diseases, electrophysiology, guided surgery, and guided biopsies. Such devices often are made up of a tracking system and a tracked object. The tracked object may be attached to an invasive device, e.g., catheter, guidewire, needle, surgical tool, electrophysiology probe, ablation tool, or other such device, that is used in a medical procedure. The tracking system provides the location of the tracked object and hence the location of the invasive device. The coordinates provided by the tracking system are often related to a coordinate system associated with a pre-acquired image or set of images, such as those obtained from a CT, MRI, or nuclear imaging scan. The positional information may then be used for moving or steering the invasive device to the desired location within the human body.

Many technologies have been used for tracking medical devices within the human body. Among them are the magnetic tracker, the impedance tracker and the radioactive tracker. The following brief overview of tracking technologies provides some terminology and background context. Many well-known variations of these basic technologies have been developed. See, for example, the presentation entitled Tracking: Beyond 15 Minutes of Thought by G. Bishop, G. Welch and B. Allen, available at: http://cs.unc.edu/˜tracker/ref/s2001/tracker/index.html.

### Magnetic Trackers

Magnetic trackers, a type of electromagnetic tracker, are described in U.S. Pat. No. 4,054,881, to Raab (assigned to The Austin Company) and in the article: Magnetic Position and Orientation Tracking System by F. Raab, E. Blood, T. Steiner, and H. Jones, published in the IEEE Transactions on Aerospace and Electronic Systems, Vol. 15, No. 5, 1979, pp. 709-718. These trackers employ a DC or AC magnetic field created by an antenna or a rotating magnet located outside the patient. The spatial dependence of this field is known either analytically or using an experimentally-determined field map. A probe introduced into the patient is used to measure the magnetic field in-vivo. In some other implementations, a probe that is introduced into the patient creates the magnetic field and a receiving antenna is placed outside the patient's body, or there may be both internal and external probes. The amplitude and/or phase of the measured magnetic field may be used to calculate the position of or orientation of the probe.

Magnetic trackers are relatively accurate and fast. However, they utilize a relatively large sensor or transmitter, typically of diameter at least 1 mm, that must be embedded within the tracked device. This limitation prevents magnetic trackers from being used in small devices or in small spaces such as small blood vessels. In addition, the accuracy of some magnetic trackers is degraded when metallic objects are present in the nearby environment of the tracker. Since such metallic objects are omnipresent in a typical hospital environment, the applicability of such magnetic trackers in a medical environment is limited.

### Impedance Trackers

Impedance trackers are described in U.S. Pat. No. 5,983,126, to Wittkampf (assigned to Medtronic, Inc); U.S. Pat. No. 5,697,377, to Wittkampf (assigned to Medtronic, Inc); U.S. Pat. No. 7,263,397, to Hauck et. al. (assigned to St. Jude Medical); and Published U.S. application 2007/0060833, to Hauck. Pairs of electrodes, known as source/sink electrodes or drive electrodes, are placed on the outside of the body along three roughly orthogonal axes—e.g. superior-inferior, anterior-posterior, and right-left. These electrodes create electric potential gradients throughout the body. An intra-body probe, also known as a mapping or sense electrode, measures the electric potential at a specific point. Knowing the dependence of the electric potential on position, the position of the probe may then be calculated. Additional calibration electrodes may be introduced into the body along with the sense electrode, to calibrate the electric potential gradient.

### Radioactive Trackers

Exemplary radioactive trackers are described in Published PCT Application Nos. WO 2006/016368 and WO 2007/017846, both assigned to Navotek Medical Ltd. Radioactive trackers track the position of tiny radioactive markers that emit gamma rays. In some embodiments, the tracking system is made up of a set of collimated, differential, gamma-ray sensors placed around the patient. By comparing ray counts at the two sides of each differential sensor, each such sensor may be rotated to point at the source. In another embodiment, the differential ray counts are used to calculate the angular offset between the source and the mid-plane of the specific differential sensor. In either case, each sensor provides the orientation of a plane containing the radioactive source and the sensor. If a sufficient number of sensors are used (at least three for 3D localization), the intersection or nearest-intersection of these planes may define the position of the radioactive source.

### Tracker Characteristics

Accuracy, precision, and latency are characteristics used in comparing trackers. The term accuracy means the degree of conformity of the measured or calculated position to its actual true or physical value. The term precision, also called reproducibility or repeatability, means the degree to which further measurements or calculations show the same or similar results. The term latency means the delay between the time when the tracked object begins to move and the time when this movement is detected by the tracker. A low latency tracker is referred to as “fast”, “responsive” or “having good dynamic response;” a high latency tracker is referred to as “slow”, “sluggish” or “having poor dynamic response.”

### SUMMARY OF THE INVENTION

An aspect of some embodiments of the invention concerns a hybrid tracker in which data from a radioactive tracker is used in combination with data from an impedance tracker to estimate a position of a target.

There is thus provided, in accordance with an exemplary embodiment of the invention, a hybrid radioactive and impedance tracking system for tracking a target in a human or animal body, comprising:
a) a radioactive tracking subsystem for tracking the target;
b) an impedance tracking subsystem for tracking the target; and
c) a processor that uses data from the radioactive tracking subsystem in combination with data from the impedance tracking system to estimate a position of the target.

Optionally, the hybrid tracking system also comprises a breathing cycle monitor for determining a breathing cycle phase of the body, wherein the processor uses the data to estimate the position of the target according to the breathing cycle phase.

Additionally or alternatively, the hybrid tracking system comprises a cardiac phase monitor for determining a cardiac cycle phase of the body, wherein the processor uses the data to estimate the position of the target according to the cardiac phase.

Optionally, the processor uses radioactive tracking data acquired at different times, taking into account the time at which the data was acquired, to estimate the position of the target.

Optionally, the processor estimates the position of the target using a recursive filter with dynamically changing parameters.

In an embodiment of the invention, the processor uses the radioactive tracking data to correct an error in a position of the target estimated from the impedance tracking data.

There is further provided, in accordance with an exemplary embodiment of the invention, medical system comprising:
a) a hybrid tracking system according to an exemplary embodiment of the invention; and
b) an invasive medical device comprising a target usable by the hybrid tracking system.

Optionally, the invasive device comprises one or both of a catheter and a guidewire.

Additionally or alternatively, the invasive device comprises an ablating electrode.

Optionally, the invasive device is capable of puncturing a membrane of the body.

Optionally, the invasive device comprises one or both of a biopsy needle and a locator needle.

Optionally, the invasive device comprises one or more of a stent, a balloon, and an implant coil.

There is further provided, in accordance with an exemplary embodiment of the invention, a method of tracking a target in a human or animal body, comprising:
a) repeatedly acquiring impedance tracking data of the target and using the impedance tracking data to repeatedly determine a location of the target, using an impedance tracking model;
b) acquiring radioactive tracking data of the target;
c) updating the impedance tracking model for the target, directly or indirectly using the radioactive tracking data and the impedance tracking data; and
d) repeating (a) through (c) at least once, using the updated impedance tracking model.

Optionally, when repeating (c), both earlier and later acquired tracking data is used, taking into account the different acquisition times.

Optionally, taking into account the different acquisition times comprises using a recursive filter.

Optionally, the method also comprises determining a breathing phase of the body, wherein updating the impedance tracking model comprising updating the model taking into account the breathing phase.

Additionally or alternatively, the method also comprises determining a cardiac phase of the body, wherein updating the impedance tracking model comprising updating the model taking into account the cardiac phase.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic view of a hybrid tracker system being used to track a catheter in a patient, according to an exemplary embodiment of the invention;

FIG. 2 is a schematic plot of positions and errors in position using a simulation of a hybrid tracker system according to an exemplary embodiment of the invention;

FIG. 3 is a schematic detailed view of the distal end of the catheter with tracking target shown in FIG. 1; and

FIG. 4 is a flow diagram of a method used to find a position of a target using a hybrid tracker system, according to an exemplary embodiment of the invention.

### DESCRIPTION OF EMBODIMENTS OF THE INVENTION

The present invention, in some embodiments thereof, relates to a hybrid radioactive and impedance tracker and, more particularly, but not exclusively, to a hybrid tracker used to track the position of a medical device in the body.

An aspect of some embodiments of the invention concerns a hybrid tracking system, comprising a radioactive tracking subsystem, and an impedance tracking subsystem, which track the position of a target in a human or animal body, for example a target associated with or embedded in an invasive medical device. A data processor directly or indirectly uses data from the radioactive tracking subsystem and from the impedance tracking subsystem to estimate a position of the target. Optionally, data from the radioactive tracking subsystem is used to correct errors in position of the target found by the impedance tracking subsystem. Alternatively, a position of the target is estimated by taking an average, possibly a weighted average, of a position determined by data from the radioactive tracking subsystem and a position determined by data from the impedance tracking subsystem. Alternatively, a position determined by data from the radioactive tracking subsystem is used to verify the accuracy of a position determined by data from the impedance tracking subsystem, when the two positions are in sufficiently good agreement.

In some embodiments of the invention, a target position found from the radioactive tracking data is compared to a target position found from the impedance tracking data, and a discrepancy between them is used to update the values of parameters of an impedance tracking model, which relates impedance data to the position of the target. This procedure is potentially advantageous, because impedance tracking data for locating a target generally can be acquired with relatively short latency time, and the calculated target position is repeatable at least for the short term, for example for several minutes, but is subject to relatively large systematic errors, due to spatial variations and slow temporal variations in impedance of the body. Radioactive tracking to locate a target, on the other hand, has a relatively long latency time in some embodiments, but gives the position of the target with greater absolute accuracy than impedance tracking, correlated for example to an external coordinate system. The hybrid system may combine a relatively fast dynamic response characteristic of impedance tracking with a relatively high absolute accuracy characteristic of radioactive tracking.

The long latency time of some radioactive trackers is due to the random nature of radioactivity, and the relatively low activity of the radioactive sources typically used. The signal-to-noise ratio (SNR) of some radioactive trackers is primarily determined by the Poisson statistics of radioactive decay. As a result, many photon counts may be averaged to improve the SNR in these radioactive trackers, increasing the latency of the tracking system.

The difficulty that impedance trackers may have in finding the absolute position of a target is described by Wittkampf et al, “New Technique for Real-Time 3-Dimensional Localization of Regular Intracardiac Electrodes,” Circulation 99, 1312-1317 (1999). It is often said that impedance trackers provide a repeatable location in a “deformed” space. Finding an accurate absolute position using an impedance tracker requires accurate modeling of the electric potential gradient produced inside the body by the source electrodes, but Wittkampf et al find that the gradient changes by 8 to 14% at three different positions in the left ventricle. U.S. Pat. No. 7,263,397, to Hauck et al, notes that both cardiac motion and respiratory motion cause the electric potential to change even when the measurement electrode is stationary. If this effect is not taken into account, the tracking accuracy may be significantly degraded. U.S. published patent application 2007/0060833, to Hauck, notes that the impedance of body tissues also undergoes slow changes over time, “attributable to changes in cell chemistry, for example, due to saline or other hydration drips in the patient, dehydration, or changes in body temperature.” These temporal changes, if they are not measured and taken into account in an impedance tracking model, may also degrade the accuracy of impedance tracking, making it not even repeatable over a long enough time, leading to errors of 1 centimeter or more, corresponding to errors of a few percent in the impedance.

Optionally, the radioactive tracking data is time-filtered, for example using a recursive filter such as a Kalman filter, when updating the parameters of the impedance tracking model. Optionally, the impedance tracking model, and/or the process of updating it, also takes into account information that is acquired about a phase of the breathing cycle, and/or a phase of the cardiac cycle, since the breathing cycle and the cardiac cycle can contribute to systematic errors in a target position calculated from the impedance tracking data. The impedance tracking model may include the breathing phase and/or the cardiac phase as parameters, for example.

The hybrid tracking system is optionally used to track any of a variety of invasive medical devices, used for example for therapy and/or diagnosis. The medical device optionally includes one or more of a catheter, a guide wire, an ablating electrode, an element capable of puncturing a membrane, a biopsy needle, a locator needle, a stent, a balloon, and an implant coil. In the case of an implant, the target may be embedded in the implant or in a delivery device.

In an exemplary embodiment of the invention, the target comprises both a radioactive source, used by the radioactive tracking subsystem, and at least one impedance sense electrode, which detects signals generated by the impedance tracking subsystem. The radioactive source is, for example, a gamma ray source, which optionally emits gamma rays with sufficient energy so that most of them exit the body. The radioactive tracking subsystem optionally comprises at least three differential radiation sensors, which are used, for example, to find three spatial coordinates of the source location. The impedance tracking subsystem optionally comprises one or more impedance generating sources, such as pairs of opposing electrodes placed on the skin of the body at different locations, which generate an electrical signal, for example a sinusoidal voltage or current, which is detected by the impedance sense electrode in the target, and used to calculate a position of the target. Optionally, three pairs of electrodes (at least four separate electrodes) are used, oriented in different directions in space, which can allow the impedance tracking subsystem to locate the target in three dimensions.

The radioactive source and impedance sense electrode need not be attached to the same device, but, for example, one of them could be on an implant and the other one on a delivery device for the implant, which may be kept together until the implant is delivered. The combination of the radioactive source and impedance sense electrode is still referred to herein as “a target” in this case.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

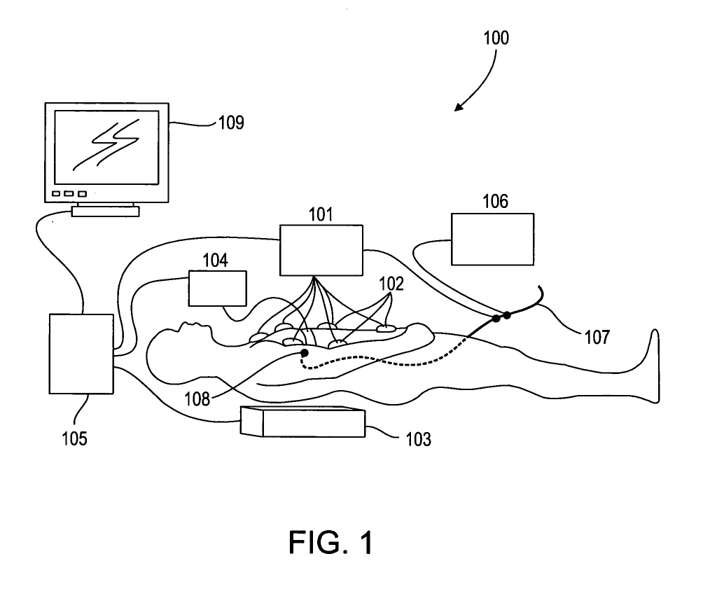

Referring now to the drawings, FIG. 1 schematically illustrates an exemplary hybrid medical tracking system 100. System 100 comprises an impedance tracking subsystem 101 with surface impedance signal source electrodes 102, used to track one or more impedance sense electrodes in a target 108. A radioactive tracking subsystem 103 is configured to measure radioactive emission from a radioactive source component of tracked target 108, optionally located at the distal end of a catheter 107, or in another invasive medical device. System 100 also optionally comprises a breathing and/or cardiac cycle tracker 104. A processor 105 combines calculated positions of the target from impedance tracking subsystem 101 and the radioactive tracking subsystem 103, and optionally also respiration and/or cardiac information from breathing/cardiac cycle tracker 104, and determines a location of tracked element 108. A hybridization algorithm, described below, is accessible to processor 105.

System 100 optionally also comprises a display 106, configured to allow visualization of the position of target 108. Display 106 is depicted in FIG. 1 as a visual display, other devices configured to implement the clinical application associated with the tracked medical device may also be included, for example processors for processing and displaying electrophysiology data. In FIG. 1, the invasive device comprises a catheter. The invasive device may also comprise other suitable devices such as guidewires or biopsy needles or the like. The solid line shows catheter 107 outside the body, and the dashed line shows the position of catheter 107 inside the body. A user interface 109 for the hybrid tracking system is also optionally included in system 100.

### Exemplary Method of Use

Processor 105 uses an impedance tracking model to relate data from the one or more impedance sense electrodes to the position of the target. For example, if the signal source electrodes comprised two large parallel planar electrodes with potential V₁ and V₂ respectively, much larger in diameter than a distance d between them, and if body tissue filling the space between them had uniform impedance, then the distance X of a sense electrode from the electrode with potential V₁ would be given by (V_{S}−V₁)d/(V₂−V₁), where V_{S} is the potential of the sense electrode. In practice, with a more complicated body and electrode geometry, and with different parts of the body having very different values of impedance, the impedance tracking model will be more complicated, optionally using a finite element model, for example, to calculate three spatial coordinates of the position of the one or more sense electrodes, as a function of their voltage relative to different source electrodes. An example of a suitable impedance tracking subsystem is the system described in U.S. Pat. No. 5,697,377, assigned to Medtronic, which uses three pairs of source electrodes producing electric fields oriented approximately in three orthogonal directions, at frequencies of 30, 31, and 32 kHz respectively, and currents of 0.1 mA each. The voltage on the sense electrode is digitized at a sufficiently fast rate to detect and reliably distinguish between the three signals. Medtronic-Cardiorhythm, of Minneapolis, Minn., USA, makes an impedance tracking system, the LocaLisa (described for example at http://localisa.com/products/localisa/index.html, downloaded on Apr. 6, 2009), similar to the one described in U.S. Pat. No. 5,697,377, and various other commercially available medical impedance tracking systems could also be used. However, the calculated target position need not be time-averaged over periods of 10 seconds, as described in U.S. Pat. No. 5,697,377, but may be found dynamically at much shorter time intervals, for example less than 0.1 seconds, less than 0.03 seconds, less than 0.01 seconds, less than 0.003 seconds, or less than 0.001 seconds, or less than 0.0003 seconds. The hybrid tracking system may optionally be used to greatest advantage if the impedance tracking subsystem is used to calculate target positions much more frequently than the radioactive tracking subsystem.

Optionally, the impedance tracking model is initially based on an average patient's body. However, it has been found that even the best a priori impedance models may produce errors of 10% or more in the electric field at a given location, and hence in the calculated position of the target. Furthermore, the error may vary from place to place in a way that is difficult to predict, and may vary in time, due to the respiratory and cardiac cycles, but also including relatively slow temporal variations of a few percent or more that are difficult to model or predict. Possibly, to overcome these problems, and/or to avoid the need to develop an accurate patient-specific impedance model, the impedance tracking model optionally includes some free parameters, with values that can be adjusted by comparing the calculated target position with the generally more accurate target position obtained from the radioactive tracking subsystem. For example, the free parameters can include a correction in position for each of the three spatial coordinates, and/or a correction in the derivative of each of the three spatial coordinates with respect to a potential difference between a sense electrode and each of the source electrodes. Higher derivatives may also optionally be included, as additional free parameters. It should be noted that signals from the different source electrodes can be activated at different times, or a different frequencies, so that they do not interfere with each other, and/or can be distinguished by the one or more sense electrodes. These free parameters would be expected to vary widely at different locations in the body, but since they are frequently updated, as the target moves through the body, there may be no need to have a globally accurate impedance model in advance.

In an exemplary embodiment of the invention, processor 105 uses data from the radioactive tracking subsystem to update the values of the free parameters of the impedance tracking model. Optionally, this is used to reduce errors in the position of the target calculated by the impedance tracking model from the impedance tracking data. The radioactive tracking subsystem generally has a much longer latency time than the impedance tracking system, for example requiring 0.1 seconds, or 0.3 seconds, or 1 second, to acquire data for an updated target position. But optionally, the calculated target position is more accurate, absolutely, than the target position calculated by the impedance tracking subsystem, without the relatively large systematic errors of the impedance tracking subsystem. For example, the radioactive tracking subsystem may give a target position that is accurate to within 1 mm, with respect to an external coordinate system. An example of a suitable radioactive tracking subsystem is a radioactive tracking system described in published PCT application WO2007/017846, assigned to Navotek Medical, Ltd. Using a source of 50 to 100 μCi of ¹⁹²Ir, spatial resolution of better than 1 mm can be obtained, with a latency of 0.1 to 1 second.

In some embodiments of the invention, in order to update the free parameters of the impedance tracking model, differences are found at different times between the target position found by the radioactive tracking subsystem, and the position calculated from the impedance tracking data, and the differences are used to update the free parameters of the impedance tracking model. Optionally, the data is time filtered, optionally to take into account the generally longer latency time of the radioactive tracking data, and the noise in both calculated target positions. For example, a recursive filter, such as a Kalman filter, is used when updating the parameters of the impedance tracking model.

In a Kalman filter, a system is described mathematically, at least in part, by a “state vector”. A model equation predicts how the state vector evolves from one discrete time step to the next time step. This model is known as the state transition model. The state vector is initialized to some value and the state-transition equations are then used to predict the value of the state vector one time step ahead at a specified time. At some time step, a measured quantity becomes available. In using a Kalman filter, these measured values must have a known, analytic relationship to the parameters of the state vector. This relationship is known as the observation model. At the time each measured value becomes available, two estimates of the state vector are produced: one is provided by the prediction from the previous step and one is derived from the measured quantities. The equations of the Kalman filter perform an optimal combination of these two estimates and calculate the accuracy of this optimal estimation.

### Simulation Model of Hybrid Tracking System

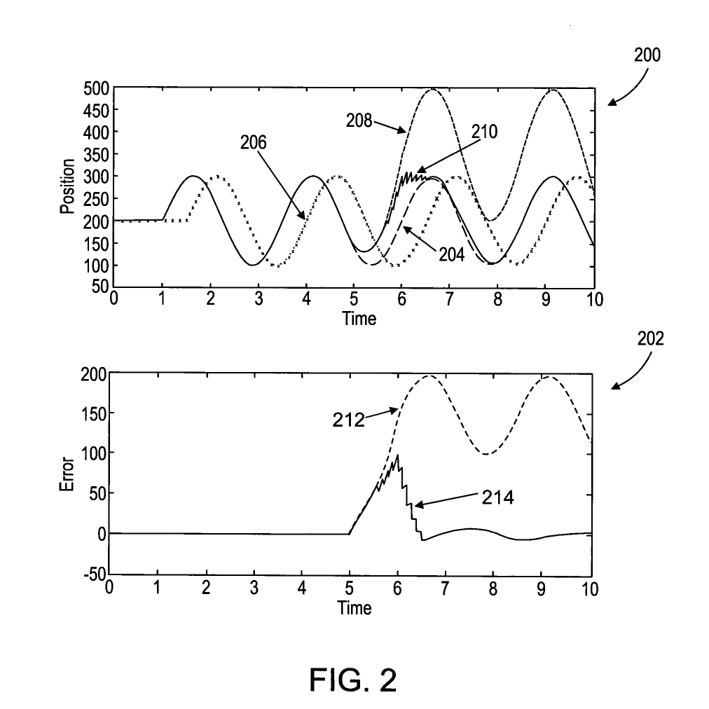

FIG. 2 shows a plot 200 of the position of a target as a function of time, and a plot 202 of the error in the position, using a simulated hybrid tracking system and a simple impedance tracking model, to illustrate how the hybrid tracking system can reduce errors in the calculated position of the target. In this simulation, only a single coordinate X of the target position is tracked. The actual target position 204 is plotted as a curve with long dashes in plot 200, although in the part of the curve at t<5, where the calculated position matches the actual position, curve 204 looks like a solid line because it overlaps the curve showing the calculated position. From t=0 to t=1, the target remains stationary at X=200, and for the rest of the simulation, from t=1 to t=10, the target oscillates sinusoidally between X=100 and X=300. The target position 206 calculated from the radioactive tracking data, shown as a series of small crosses, has a latency of 0.5, so lags behind the actual target position, but otherwise finds the position accurately.

Between t=0 and t=5, the voltage of the sense electrode of the impedance tracking subsystem is assumed to be V=k₁X+k₀, where k₁ and k₀ are constants. The impedance model is initially set at X=(V−K₀)/K₁, with the same values of K₀=k₀ and K₁=k₁, so the target position X calculated by the impedance tracking subsystem, indicated by a curve 208 with short dashes, accurately reflects the actual target position. The radioactive tracking subsystem gives the same target position, taking into account the latency time of 0.5, so the impedance model does not change, and the hybrid system gives the same target position X as the impedance tracking subsystem.

Starting at t=5, the values of k₀ and k₁ are both suddenly increased by 50%. Since the impedance model initially remains unchanged, using the old values of the free parameters K₀ and K₁, the target position X calculated by the impedance tracking subsystem, indicated by curve 208, starts to diverge from the actual target position, indicated by curve 204. At t=5.5, after the latency time for the radioactive tracking subsystem has passed, it becomes apparent that the radioactive tracking data is inconsistent with the target position calculated from the impedance tracking data. Every time a new calculated target position is available from the radioactive tracking data, indicated by a small cross on curve 206, an adjustment is made in the values of the free parameters K₀ and K₁ used in the impedance tracking model. Because the data is time-filtered in this example, using a Kalman filter, in updating the impedance tracking model, the impedance model is not instantaneously adjusted to match the new correct values of k₀ and k₁ assumed in the simulation, but the free parameters K₀ and K₁ in the impedance tracking model gradually approach these values during the rest of the simulation, as the hybrid system “learns” the correct values. The calculated target position 210 from the hybrid system, calculated from the impedance tracking data using the currently updated impedance tracking model, gradually approaches the true target position 204, becoming quite accurate by t=6.5. It should be noted that the position 210 calculated by the hybrid tracking system, unlike the position 206 calculated from the radioactive tracking system, does not have any latency, but instantaneously tracks the target as long as k₀ and k₁ are not making sudden changes. Plot 202 shows the error 212 in the target position found from the impedance tracking data using the initial impedance tracking model, which never is corrected after t=5, and the error 214 in the target position found by the hybrid tracking system, which uses the radioactive tracking data to correct the impedance tracking model. Error 214, after initially matching error 212, quickly becomes much smaller as the radioactive tracking data is taken into account.

### Exemplary Tracked Target

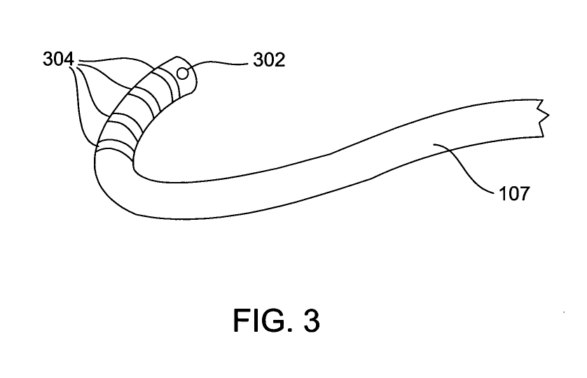

FIG. 3 shows further details of the distal end of catheter 107, according to an exemplary embodiment of the invention. In particular, FIG. 3 shows a compact design of the tracked target comprising a radioactive source 302 and a plurality of impedance sense electrodes 304. Conductors such as wires or ribbons, not shown, optionally run through or along the catheter, carrying detected impedance signals from the impedance sense electrodes 304 to impedance tracking system 101 shown in FIG. 1. Signals from the impedance sense electrodes may also be transferred out of the body using wireless communication.

A suitable radioactive source for most sites in the human body has an activity in the range of 2 to 200 μCi and emits gamma radiation with energy in the range of 50 to 700 KeV. Exemplary isotopes are ⁵⁷Co, ¹⁹²Ir and ¹³³Ba.

The one or more impedance sense electrodes measure the voltage at one or more locations in the body. When used on an elongated tool such as a catheter or guidewire, a number of electrodes measuring voltage at a number of distinct locations may be placed along the catheter (or other tool). Additional electrodes are also optionally employed in the catheter to measure the gradient of the electric potential. Additional electrodes may also be added to track changes in the shape and orientation of the catheter.

### Exemplary Invasive Medical Devices

The hybrid tracking system may be used to track invasive medical devices, for example intravascular tools such as guidewires or catheters. The hybrid tracking system is also potentially useful for tracking with medical devices that puncture one or more membranes of the human body, for example biopsy needles or locator needles for directing medication to a particular treatment site. Although intravascular catheters will be used as convenient examples for explaining the utility and operation of the hybrid tracking system, it should be understood that the hybrid tracking system may also be suitable for use with other procedures or devices for which real time knowledge of the position of a tracked element would be useful.

The hybrid tracking system may be used in association with catheters used in intravascular treatments such as stent placement, electrophysiology mapping or treatment (e.g., ablation), treatment of aneurisms with devices such as stents, balloons, GDC or other coils, and localized intravascular drug delivery. As applicable, a medical system utilizing the hybrid tracking system may comprise one or more appropriate elements in addition to a tracked catheter, including various allied components and implants such as stents, balloons, sensing and ablating electrodes, coils, drug delivery tubes, and the like.

### Exemplary Radioactive Tracking Subsystem

The radioactive tracking subsystem optionally comprises a set of at least three sensors each acting as a differential gamma radiation detector. The subsystem may also comprise signal processors for processing measurements taken by the sensors. Methods for detecting gamma rays are well known to those skilled in the art of photon detection. See, for example, the book Radiation Detection and Measurement by Glenn Knoll, 3rd. edition (2000), ISBN 0-471-07338-5. The radiation detectors may comprise thallium-doped cesium iodide (CsI(Tl)) scintillation crystals coupled to photo multiplier tubes (PMT). Additionally or alternatively, the radiation detectors may comprise solid state detectors such as cadmium-zinc-telluride (CZT) detectors or other detection devices suitable for detecting radiation emitted by the chosen radioactive source.

Each differential radiation sensor optionally measures an angular offset towards the tracked radiation source, thus defining a plane in which the source lies. A radioactive tracker processor is used to calculate the location of the radioactive source by calculating the intersection or nearest-intersection of the at least three planes defined by the at least three sensors. The radioactive tracker may operate at a low sampling rate, e.g., at about 5 to 25 Hz, for example at 10 Hz. At such low sampling rates and. long sampling times, there is no need to employ a low pass filter to improve the SNR of the radioactive tracker subsystem.

### Exemplary Impedance Tracking Subsystem

The impedance tracking subsystem comprises a number of drive electrodes for imposing a signal upon the body region under study, one or more signal generators for producing those signals, and one or more processors for determining the position of impedance sense electrodes based upon voltages measured at the impedance sense electrodes and at other electrodes.

Many existing impedance tracking systems employ or comprise at least six drive electrodes. The electrodes are typically placed on the patient's skin roughly along the three main body axes. Such drive systems also include circuitry that drives current between selected pairs of drive electrodes. For example, the system may drive an alternating current between pairs of opposite electrodes. The system optionally imposes an alternating current at a distinct frequency on each pair of drive electrodes. The processors determine the location of each impedance sense electrode based upon the voltage measured at that impedance sense electrode. The processors may employ a frequency separating algorithm, for example, a discrete Fourier transform, to discriminate amongst the voltage measurements that correspond to each pair of drive electrodes. Alternatively, the impedance tracker subsystem may drive all the currents at a single frequency or at DC and utilize a time sharing mechanism for distinguishing among the pairs of drive electrodes.

The impedance tracking subsystem optionally comprises one or more processors employed to calculate the location of each of the impedance sense electrodes using impedance gradients. For instance, if a catheter employing multiple sense electrodes is employed, the signals from those electrodes may be used to calculate the shape and/or orientation of the tip of the catheter, by finding the position of each electrode, for example. Additionally or alternatively, multiple sense electrodes may be used to calibrate potential gradients. The inventors have found that a refresh rate of the impedance tracking subsystem in the range of 100 Hz to 2 KHz is very useful in the hybrid tracking system.

The impedance tracker processor functions need not be performed by dedicated processors located in a separate unit, as possibly suggested by FIG. 1, but optionally are performed by a computer or other data processing system also used for other functions, such as finding the location of the target from the radioactive tracking data, and updating the impedance tracking model using the radioactive tracking data.

The hybrid tracking system comprises one or more processors for performing calculations relating to combining (“hybridizing”) the outputs of the radioactive tracking subsystem and the impedance tracking subsystem in calculating the real-time location of the tracked target. The processors may comprise one or more digital general purpose computers (e.g., PC's), specialized computers (e.g., DSP's), or semi-specialized computers (e.g., Z-80 CPU's and supporting devices) including software, hardware, firmware, or mixtures that include programs, incorporating a hybridization algorithm such as discussed below.

The impedance tracking subsystem generally responds relatively quickly to movements of the tracked target and has a low noise level. However, its coordinate system is deformed, with systematic errors that vary in space, and may also vary slowly in time. The radioactive tracking subsystem generally has greater absolute accuracy in calculating the location of the tracked target in a real-world coordinate system. However, it generally responds more slowly to movement of the target.

### Exemplary Algorithm Used by Hybrid Tracking System

As will be explained below, in an exemplary embodiment of the invention the hybridization algorithm generates an impedance tracking model of the “deformed” impedance tracking coordinate system, and updates this model at a relatively slow rate based on the data of the radioactive tracking subsystem. The real time tracking of the tracked target is based on the data of the impedance tracking system, using this deformation model that is repeatedly updated by the data of the radioactive tracker subsystem.

In some embodiments of the hybridization algorithm uses a time filter, for example a recursive filter such as a Kalman filter, in which a changing state vector includes free calibration parameters of the impedance tracking deformation model, needed to translate the voltage readings of the one or more impedance sensor electrodes into coordinate values. In some embodiments of the invention, these parameters include the derivative of each coordinate value with respect to one of the voltage readings, i.e., three parameters; in other embodiments of the invention, these parameters may include the derivative and offset of each coordinate value with respect to one of the voltage readings, i.e., six parameters. Additional parameters may also be used, for example representing partial derivatives of the coordinate values with respect to different voltage readings, to take into account the possibility that the electric fields generated by the pairs of electrodes are rotated with respect to the coordinate system. Parameters representing second and higher derivatives of the coordinate values with respect to the voltage readings may also be used. The calibration parameters are repeatedly updated, as new radioactive tracking data becomes available from the radioactive tracking system. In the interim between updates of the calibration parameters, the impedance tracker continues to provide position measurements using the current value of the parameters.

It should be understood that whenever the term “Kalman filter” is referred to herein, any recursive filter with dynamically changing parameters that combines two sources of information can generally be used instead. Such filters are not limited to Kalman's original formulation, but include, for example, any of the variations on Kalman filters described in the Wikipedia article on Kalman filters, downloaded from <http://en.wikipedia.org/wiki/Kalman_filter> on Apr. 3, 2009, including the Stratanovich filter, the information filter, the fixed-lag smoother, the extended Kalman filter, the unscented Kalman filter, and the Kalman-Bucy filter. Techniques of linear and non-linear Kalman filtering are also described, for example, in the article A New Approach to Linear Filtering and Prediction Problems by R. E. Kalman, published in the Transactions of the ASME—Journal of Basic Engineering Vol. 82: pp. 35-45 (1960), the article New Results in Linear Filtering and Prediction Theory by R. E. Kalman and R. S. Bucy published in the Transactions of the ASME—Journal of Basic Engineering Vol. 83: pp. 95-107 (1961), the book Applied Optimal Estimation by A. Gelb (editor), ISBN Number: 0262570483 (available from NavtechGPS, Springfield, Va.) and the book Stochastic Models, Estimation, and Control by Peter Maybeck (available from NavtechGPS).

Alternatively, any other adaptive low-pass digital filter is used.

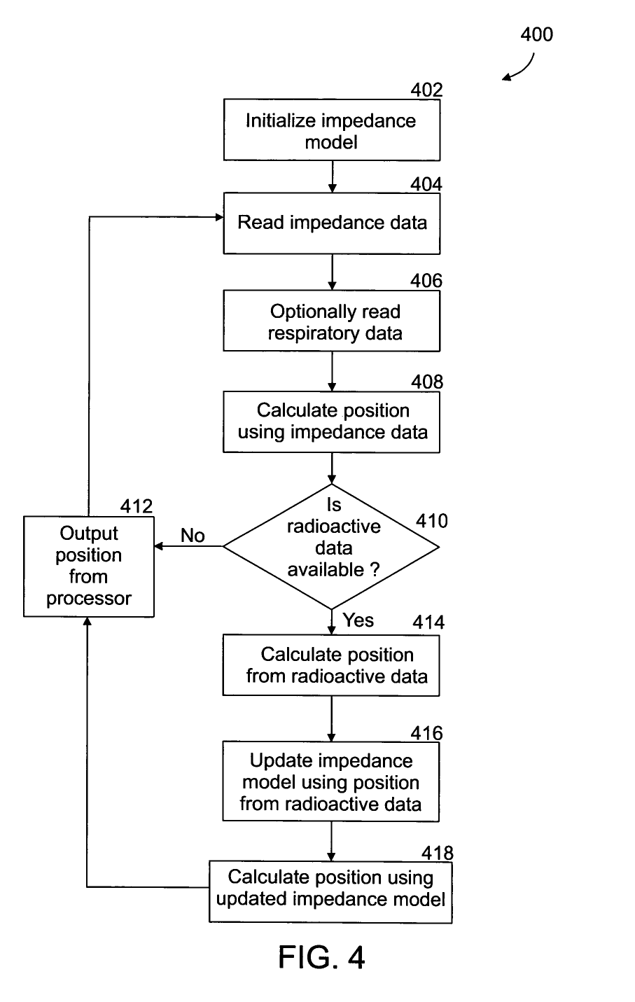

FIG. 4 shows a flow chart 400 for a method used by the hybrid tracking system in an exemplary embodiment of the invention. At 402, the calibration parameters of the impedance tracking model are initialized to default values. Specifically, the state vector of the Kalman filter is initialized to these values. For example, average values of calibration parameters found in a group of patients, for a particular region of the body where the target is initially located, may be chosen as the default values. At 404, voltage data is read from the impedance sense electrode or electrodes in the target. Optionally, respiratory phase data, and/or cardiac phase data, is acquired at 406. Cardiac phase may be measured, for example, using an ECG, or any other known method. Respiratory phase may be measured using any of the methods and devices known in the art, some of which are described below. At 408, the current calibration parameters are used to estimate the position of the target, optionally taking into account the respiratory and/or cardiac phase if it was measured or estimated at 406. At 410, a check is made whether radioactive tracking data is available. Specifically, a check is made whether sufficient radioactive tracking data has accumulated since the last time the target location was calculated from the radioactive data, in order to provide a new independent estimate of the target location with a desired precision, for example within 1 mm. If the radioactive data are not available, the calculated position of the target is outputted from the processor at 412, and control returns to 404. This loop is repeated until the radioactive data are available. If the radioactive data are available, at 414 the location of the target is calculated from the radioactive data. At 416, this data is applied, along with the impedance tracking data, in updating an estimate of the calibration parameters using the equations of the Kalman filter. Optionally, if respiratory or cardiac phase data have been acquired, this data is also used in updating the calibration parameters, for example by using an impedance tracking model in which the calibration parameters are functions of the respiratory and/or cardiac phase. For example, a set of 2 to 15 values of each of the calibration parameters may be used, one value for each of 2 to 15 different ranges of the cardiac and/or respiratory phase. If two values are used for different ranges of the respiratory phase, for example, one value could correspond to having the lungs mostly inflated, and the other value could correspond to having the lungs largely deflated, and if two values are used for different ranges of the cardiac phase, one value could correspond to the systole and the other value could correspond to the diastole. At 418, the updated calibration parameters are used to re-calculate the position of the target. The calculated position is outputted by the processor at 412, and control returns to 404. The resulting outputs in position of the target have the fast dynamic response of the impedance tracking subsystem, but accuracy that can approach that of the radioactive tracking subsystem.

### Methods for Measuring the Respiratory Phase

Some details about respiratory motion and its measurement may be found in The Management of Respiratory Motion in Radiation Oncology: Report of AAPM Task Group 76, available on the World Wide Web at: http://aapm.org/pubs/reports/RPT_{—}91.pdf and in the Ph.D. thesis of Laura Mason entitled “Signal Processing Methods for Non-Invasive Respiration Monitoring”, Trinity College, Oxford University, 2002. Devices suitable for monitoring and quantifying movement of the chest during a patient's respiratory phase include transthoracic inductance and impedance plethysmographs, strain gauge measurement of thoracic circumferences, pneumatic respiration and whole body plethysmographs, image-based sensors that monitor the torso either directly by reflecting a light beam off of a mirror placed on the torso or by monitoring a light source placed on the torso, capnography monitors that measure CO₂ in respiration gas using infra-red sensors, differential pressure pneumotachometers to measure airway pressure, etc. When such monitoring is performed, a “learning” or “training” phase is optionally used to determine the relationship between the monitored parameter and the respiratory phase. After the learning phase is complete, the parameter may be monitored in real time and used to estimate the relative position of the measurement in the respiratory cycle, e.g., start/end inspiration, start/end expiration, etc.

It is expected that during the life of a patent maturing from this application many relevant radioactive and impedance tracking systems will be developed and the scope of the terms radioactive tracker and impedance tracker, or tracking system, is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A hybrid radioactive and impedance tracking system for tracking a target in a human or animal body, comprising:
a) a radioactive tracking subsystem for tracking the target;
b) an impedance tracking subsystem for tracking the target; and
c) a processor that uses data from the radioactive tracking subsystem in combination with data from the impedance tracking system to estimate a position of the target.

2. A hybrid tracking system according to claim 1, also comprising a breathing cycle monitor for determining a breathing cycle phase of the body, wherein the processor uses the data to estimate the position of the target according to the breathing cycle phase.

3. A hybrid tracking system according to claim 1 or claim 2, also comprising a cardiac phase monitor for determining a cardiac cycle phase of the body, wherein the processor uses the data to estimate the position of the target according to the cardiac phase.

4. A hybrid tracking system according to any of the preceding claims, wherein the processor uses radioactive tracking data acquired at different times, taking into account the time at which the data was acquired, to estimate the position of the target.

5. A hybrid tracking system according to claim 4, wherein the processor estimates the position of the target using a recursive filter with dynamically changing parameters.

6. A hybrid tracking system according to any of the preceding claims, wherein the processor uses the radioactive tracking data to correct an error in a position of the target estimated from the impedance tracking data.

7. A medical system comprising:
a) a hybrid tracking system according to any of the preceding claims; and
b) an invasive medical device comprising a target usable by the hybrid tracking system.

8. A medical system according to claim 7, wherein the invasive device comprises one or both of a catheter and a guidewire.

9. A medical system according to claim 8, wherein the invasive device comprises an ablating electrode.

10. A medical system according to any of claims 7-9, wherein the invasive device is capable of puncturing a membrane of the body.

11. A medical system according to any of claims 7-10, wherein the invasive device comprises one or both of a biopsy needle and a locator needle.

12. A medical system according to any of claims 7-11, wherein the invasive device comprises one or more of a stent, a balloon, and an implant coil.

13. A method of tracking a target in a human or animal body, comprising:
a) repeatedly acquiring impedance tracking data of the target and using the impedance tracking data to repeatedly determine a location of the target, using an impedance tracking model;
b) acquiring radioactive tracking data of the target;
c) updating the impedance tracking model for the target, directly or indirectly using the radioactive tracking data and the impedance tracking data; and
d) repeating (a) through (c) at least once, using the updated impedance tracking model.

14. A method according to claim 13, wherein, when repeating (c), both earlier and later acquired tracking data is used, taking into account the different acquisition times.

15. A method according to claim 14, wherein taking into account the different acquisition times comprises using a recursive filter.

16. A method according to any of claims 13-15, also comprising determining a breathing phase of the body, wherein updating the impedance tracking model comprising updating the model taking into account the breathing phase.

17. A method according to any of claims 13-16, also comprising determining a cardiac phase of the body, wherein updating the impedance tracking model comprising updating the model taking into account the cardiac phase.
